(19) United States
(12) Patent Application Publication
(10) Pub. No.: US 2025/0360252 A1
(43) Pub. Date: Nov. 27, 2025

(54) THERMAL MANAGEMENT FOR AN OXYGENATOR DEVICE

(71) Applicant: Abiomed, Inc., Danvers, MA (US)

(72) Inventors: Brian Wenholz, Danvers, MA (US); Thomas Underwood, Danvers, MA (US); Gregory Herwig, Danvers, MA (US); Arkadiy Tsitlik, Danvers, MA (US); Stephen Cournane, Danvers, MA (US); Amar Vadalia, Danvers, MA (US)

(21) Appl. No.: 19/210,460

(22) Filed: May 16, 2025

Related U.S. Application Data
(60) Provisional application No. 63/650,462, filed on May 22, 2024.

(51) Int. Cl. A61M 1/16

(52) U.S. Cl. CPC A61M 1/1603; A61M 2202/0208; A61M 2205/07; A61M 2205/3334; A61M 2205/3368

(57) ABSTRACT

Methods and apparatus for controlling a flow rate of an oxygenator device are described. The method includes receiving a first temperature associated with a blower of the oxygenator device, wherein the first temperature is sensed by a temperature sensor of the oxygenator device, and adjusting a flow rate of oxygen provided to a blood oxygenator by the blower based, at least in part, on the first temperature.

### RELATED APPLICATIONS

This application claims the benefit under 35 U.S.C. § 119(e) to U.S. Provisional Patent Application No. 63/650,462, filed May 22, 2024, and titled, “THERMAL MANAGEMENT FOR AN OXYGENATOR DEVICE,” the entire contents of which is incorporated by reference herein.

### TECHNICAL FIELD

This disclosure relates to techniques for managing thermal properties of an oxygenator device.

### BACKGROUND

Oxygenator devices, such as membrane oxygenator devices, are often incorporated into cardiopulmonary bypass (CPB) systems or extracorporeal membrane oxygenation (ECMO) systems. CPB is a rather short-term technique to take over the function of the heart and lungs during heart surgery, e.g. coronary bypass heart surgery, whereas ECMO can be used for longer-term treatments of a range of cardiac and pulmonary dysfunctions and for recovery. The function of the oxygenator device in CPB and ECMO systems is to add oxygen to a patient's blood and to remove carbon dioxide from the blood to mimic or assist the function of the patient's lungs, e.g., to allow the exchange of oxygen and carbon dioxide between the patient's blood and a gas phase inside the oxygenator device at levels in a physiological range. In a membrane oxygenator, exchange of oxygen and carbon dioxide is accomplished via a semipermeable membrane which separates the blood side from the gas side inside the oxygenator device, while enabling the diffusion of oxygen and carbon dioxide across/through the membrane. Carbon dioxide in the blood, in particular in the central arterial blood, plays a critical role in the regulation of a variety of physiological functions such as respiratory rates and reflex and maintenance of a normal pH in the blood of around 7.4.

### SUMMARY

Described herein are systems and methods for managing the thermal properties of an oxygenator device during use. Patients that use oxygenator devices are often very sick and may not be capable of sufficiently oxygenating their blood and/or removing carbon dioxide from their blood on their own. Accordingly, it is important that oxygenator devices remain operational throughout their use to ensure that patients are provided with adequate support as intended by their healthcare professionals. Conventional oxygenator devices do not typically include thermal management systems configured to control the temperature of the device within a desired operating range. Accordingly, when the temperature of an oxygenator device exceeds a threshold value (e.g., the device becomes too hot), the device may enter a failure mode in which the oxygenator device fails to operate properly and/or shuts down. As described above, failure of the oxygenator device to operate properly may result in negative health consequences for the patient using the device. In some embodiments, the temperature associated with an oxygenator device is monitored and actively managed to reduce the risk of a temperature-based failure of the device, thereby improving patient care.

In some embodiments an oxygenator device is provided. The oxygenator device includes a blood oxygenator configured to add oxygen to and remove carbon dioxide from blood when present therein, a blower configured to provide the oxygen to the blood oxygenator at a flow rate, a temperature sensor configured to sense at a first time, a first temperature associated with the blower, and at least one controller configured to adjust the flow rate of oxygen provided to the blood oxygenator by the blower based, at least in part, on the first temperature.

In one aspect, the oxygenator device further includes a heat sink coupled to the blower, wherein the temperature sensor is coupled to the heat sink. In another aspect, the temperature sensor is a thermistor. In another aspect, the at least one controller is further configured to determine whether the first temperature is greater than a first threshold value, and adjust the flow rate of oxygen provided by the blower by decreasing the flow rate when it is determined that the first temperature is greater than the first threshold value. In another aspect, the temperature sensor is configured to sense at a second time, a second temperature associated with the blower, the second time being after the first time, and the at least one controller is further configured to determine whether the second temperature is less than a second threshold value, and control at least one operation of the oxygenator device when it is determined that the second temperature is less than the second threshold value. In another aspect, the first threshold value and the second threshold value are a same value. In another aspect, the second threshold value is less than the first threshold value. In another aspect, the at least one controller is further configured to control at least one operation of the oxygenator device by outputting an indication on a user interface of the oxygenator device. In another aspect, the indication includes an indication of the second temperature. In another aspect, the indication includes an indication that the flow rate can be increased. In another aspect, the at least one controller is further configured to control at least one operation of the oxygenator device by automatically increasing the flow rate. In another aspect, automatically increasing the flow rate comprises automatically increasing the flow rate to the flow rate set at the first time. In another aspect, decreasing the flow rate comprises lowering a maximum operating voltage provided to the blower.

In another aspect, the at least one controller is further configured to receive, via a user interface of the oxygenator device, a request to increase the flow rate to a new flow rate, determine whether increasing the flow rate to the new flow rate is likely to result in a temperature of the blower exceeding a first threshold value within a predetermined amount of time, and increase the flow rate to the new flow rate when it is determined that increasing the flow rate to the new flow rate is unlikely to result in the temperature of the blower exceeding the first threshold value within the predetermined amount of time. In another aspect, the at least one controller is further configured to restrict increasing the flow rate to the new flow rate when it is determined that increasing the flow rate to the new flow rate is likely to result in the temperature of the blower exceeding the first threshold value within the predetermined amount of time. In some embodiments, an extracorporeal membrane oxygenator (ECMO) system is provided that includes the oxygenator device described herein.

In some embodiments, a controller for an oxygenator device is provided. The controller is configured to receive a first temperature associated with a blower of the oxygenator device, wherein the first temperature is sensed by a temperature sensor of the oxygenator device, and adjust a flow rate of oxygen provided to a blood oxygenator by the blower based, at least in part, on the first temperature.

In one aspect, the temperature sensor is a thermistor. In another aspect, the controller is further configured to determine whether the first temperature is greater than a first threshold value, and adjust the flow rate of oxygen provided by the blower by decreasing the flow rate when it is determined that the first temperature is greater than the first threshold value. In another aspect, the first temperature is sensed by the temperature sensor at a first time, and the controller is further configured to receive a second temperature sensed by the temperature sensor at a second time, the second time being after the first time, determine whether the second temperature is less than a second threshold value, and control at least one operation of the oxygenator device when it is determined that the second temperature is less than the second threshold value. In another aspect, the first threshold value and the second threshold value are a same value. In another aspect, the second threshold value is less than the first threshold value. In another aspect, the controller is further configured to control at least one operation of the oxygenator device by outputting an indication on a user interface of the oxygenator device. In another aspect, the indication includes an indication of the second temperature. In another aspect, the indication includes an indication that the flow rate can be increased. In another aspect, the controller is further configured to control at least one operation of the oxygenator device by automatically increasing the flow rate. In another aspect, automatically increasing the flow rate comprises automatically increasing the flow rate to the flow rate set at the first time. In another aspect, decreasing the flow rate comprises lowering a maximum operating voltage provided to the blower.

In another aspect, the controller is further configured to receive, via a user interface of the oxygenator device, a request to increase the flow rate to a new flow rate, determine whether increasing the flow rate to the new flow rate is likely to result in a temperature of the blower exceeding a first threshold value within a predetermined amount of time, and increase the flow rate to the new flow rate when it is determined that increasing the flow rate to the new flow rate is unlikely to result in the temperature of the blower exceeding the first threshold value within the predetermined amount of time. In another aspect, the controller is further configured to restrict increasing the flow rate to the new flow rate when it is determined that increasing the flow rate to the new flow rate is likely to result in the temperature of the blower exceeding the first threshold value within the predetermined amount of time. In some embodiments, an extracorporeal membrane oxygenator (ECMO) system including the controller described herein is provided.

In some embodiments, a method of controlling a flow rate of an oxygenator device is provided. The method includes receiving a first temperature associated with a blower of the oxygenator device, wherein the first temperature is sensed by a temperature sensor of the oxygenator device, and adjusting a flow rate of oxygen provided to a blood oxygenator by the blower based, at least in part, on the first temperature.

In one aspect, the method further includes determining whether the first temperature is greater than a first threshold value, and adjusting the flow rate of oxygen provided by the blower by decreasing the flow rate when it is determined that the first temperature is greater than the first threshold value. In another aspect, the first temperature is received at a first time, and the method further includes receiving at a second time, a second temperature associated with the blower, the second time being after the first time, determining whether the second temperature is less than a second threshold value, and controlling at least one operation of the oxygenator device when it is determined that the second temperature is less than the second threshold value. In another aspect, the first threshold value and the second threshold value are a same value. In another aspect, the second threshold value is less than the first threshold value. In another aspect, controlling at least one operation of the oxygenator device comprises outputting an indication on a user interface of the oxygenator device. In another aspect, the indication includes an indication of the second temperature. In another aspect, the indication includes an indication that the flow rate can be increased. In another aspect, controlling at least one operation of the oxygenator device comprises automatically increasing the flow rate. In another aspect, automatically increasing the flow rate comprises automatically increasing the flow rate to the flow rate set at the first time. In another aspect, decreasing the flow rate comprises lowering a maximum operating voltage provided to the blower.

In another aspect, the method further includes receiving, via a user interface of the oxygenator device, a request to increase the flow rate to a new flow rate, determining whether increasing the flow rate to the new flow rate is likely to result in a temperature of the blower exceeding a first threshold value within a predetermined amount of time, and increasing the flow rate to the new flow rate when it is determined that increasing the flow rate to the new flow rate is unlikely to result in the temperature of the blower exceeding the first threshold value within the predetermined amount of time. In another aspect, the method further includes restricting increasing the flow rate to the new flow rate when it is determined that increasing the flow rate to the new flow rate is likely to result in the temperature of the blower exceeding the first threshold value within the predetermined amount of time.

### BRIEF DESCRIPTION OF DRAWINGS

FIG. 1 schematically illustrates an oxygenator device, in accordance with some embodiments of the present disclosure.

FIG. 2 illustrates a flowchart of a process for managing the temperature of a blower for an oxygenator device, in accordance with some embodiments of the present disclosure.

FIG. 3 illustrates a flowchart of a process for controlling the air flow rate of a blower for an oxygenator device in response to a user request, in accordance with some embodiments, of the present disclosure.

### DETAILED DESCRIPTION

Oxygenator devices used in extracorporeal membrane oxygenation (ECMO) systems function to add oxygen to a patient's blood and to remove carbon dioxide from the blood when the patient's health is such that their body cannot adequately perform these functions on its own. The inventors have recognized and appreciated that existing oxygenator devices, which typically do not include a temperature sensing and/or management system, are susceptible to failure if the devices overheat. For example, the user may be provided with a warning that the device is overheating and/or one or more seals in the oxygenator device may fail when the device becomes too hot. Additionally, when the device heats up, the blower of the device may work harder resulting in a thermal runaway condition that causes the device to shut down. Some embodiments of the present disclosure relate to a temperature-based feedback loop for an oxygenator device that can be used to control the operation of the device such that its temperature remains within desired operating temperature limits. By controlling the temperature of the oxygenator device, the downtime of the oxygenator device may be reduced and the healthcare benefits associated with use of the device may be improved.

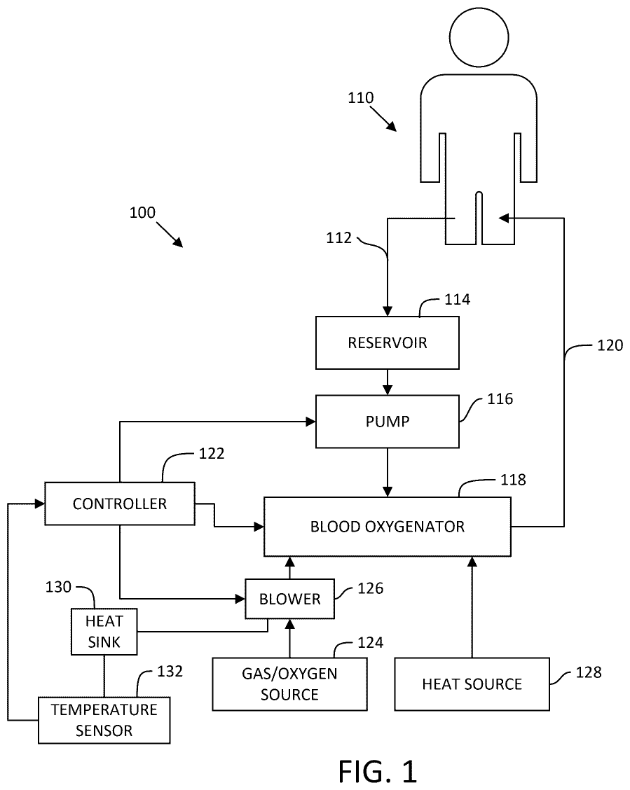

FIG. 1 schematically illustrates components of an oxygenator device 100, in accordance with some embodiments of the present disclosure. Blood taken from a patient 110 may travel through drainage cannula 112 into reservoir 114. Blood pump 116 may be configured to pump blood from reservoir 114 into blood oxygenator 118. Blood oxygenator 118 may be configured to provide oxygen to the blood and remove carbon dioxide from the blood as the blood traverses one or more channels of the blood oxygenator 118. Following processing by blood oxygenator 118, the oxygenated blood may return to the patient 110 via return cannula 120. A gas/oxygen source 124 may be configured to provide oxygen to blood oxygenator 118 via blower 126. Blower 126 may be configured to provide the oxygen from gas/oxygen source 124 to blood oxygenator 118 at a particular flow rate as set by controller 122. For instance, a user (e.g., a healthcare professional) may interact with a user interface (e.g., a graphical user interface, one or more knobs or buttons associated with the oxygenator device 100, etc.) to specify a particular flow rate for the blower 126 to provide the oxygen to the blood oxygenator 118. During operation of the oxygenator device 100, the user may adjust the flow rate of oxygen provided by the blower 126 to provide the patient 110 with a desired level of support. In some embodiments, blood oxygenator 118 may also be configured to heat the blood as it passes through the blood oxygenator 118. In such embodiments, heat may be provided to blood oxygenator 118 by heat source 128. For instance, heat source 128 may be implemented as a water-based heat circulator for providing a heat exchange function to the blood oxygenator 118.

The inventors have recognized that blower 126 is a component of oxygenator device 100 that is subject to overheating when producing higher flow rates as instructed by controller 122. Blower 126 may be coupled to heat sink 130, which may function to draw heat away from blower 126 in an effort to prevent overheating. In some embodiments, heat sink 130 may be integrated with blower 126. In some embodiments, temperature sensor 132 (e.g., a thermistor, a thermocouple, a resistance temperature detector (RTD), etc.) may be coupled to heat sink 130 to measure a temperature associated with blower 126. The temperature sensed by temperature sensor 132 may be provided to controller 122, which may adjust operation of one or more components of oxygenator device 100 using one or more of the techniques described herein. For instance, as described in more detail below, controller 122 may be configured to adjust a flow rate of oxygen provided by blower 126 to blood oxygenator 118 based, at least in part, on the sensed temperature. By decreasing the flow rate of oxygen provided by blower 126, the temperature of the blower 126 may be reduced. In this way, a temperature-based feedback loop may be used to ensure that the temperature of the blower 126 remains within an operating range that is less likely to result in failure of the device due to overheating.

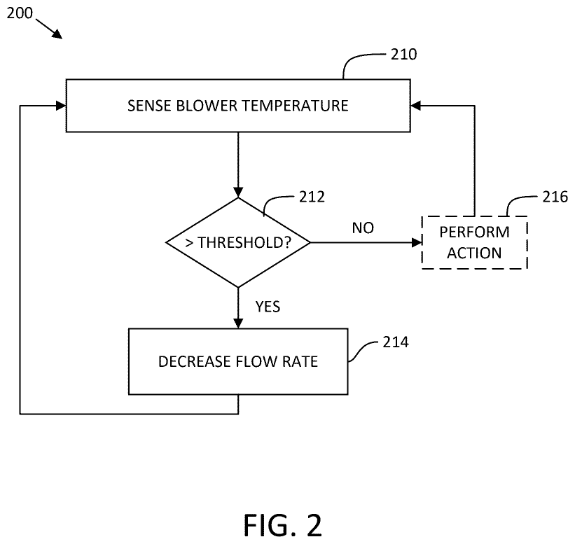

FIG. 2 illustrates a process 200 for managing the temperature of a blower of an oxygenator device, in accordance with some embodiments of the present disclosure. The inventors have recognized that in some instances, it may be possible to lower the temperature of the blower by reducing the flow rate of the blower without substantially reducing the ability of the oxygenator device to sufficiently oxygenate a patient's blood. For example, continuing to increase the flow rate of the blower above certain levels may provide only marginal additional therapeutic effects (e.g., better removal of carbon dioxide from the blood), but may increase the temperature of the blower substantially. By reducing the flow rate of the blower in such situations, the temperature of the blower may be able to be lowered without compromising the ability of the oxygenator device to provide support to the patient.

Process 200 may begin in act 210, where a temperature associated with a blower of the oxygenator device is sensed (e.g., using a temperature sensor coupled to a heat sink associated with the blower). Process 200 may then proceed to act 212, where it is determined whether the sensed temperature is greater than a threshold value. Any suitable threshold value may be used. For instance, under normal operating conditions the blower may be expected to have a temperature of 55° C. A threshold value of 60° C. may be set, and the current temperature of the blower sensed in act 210 may be compared to the threshold value. If it is determined in act 212 that the sensed temperature is greater than the threshold value, process 200 may proceed to act 214, where the flow rate of the blower may be decreased. In some embodiments, the flow rate may be adjusted based on the maximum operating voltage being provided to the blower. For instance, the flow rate may be decreased by lowering the maximum operating voltage provided to the blower. The flow rate may be decreased by any suitable amount sufficient to allow the blower to cool down. As described above, the inventors have recognized that increasing the flow rate above certain levels may produce no or marginal therapeutic benefits (e.g., increased removal of carbon dioxide from the blood) for a patient. Accordingly, in some embodiments, the flow rate of the blower may be reduced to a point where the patient is still provided with adequate support despite the lower flow rate. In some embodiments, an action may be performed in addition to decreasing the flow rate in act 214. For example, an indication (e.g., a visual indication, an audio indication, etc.) of the decreased flow rate and/or the temperature exceeding the threshold value may be provided on a user interface of the oxygenator device to alert the user to the decreased flow rate of the blower.

After decreasing the flow rate of the blower, process 200 may return to act 210 where a new temperature associated with the blower of the oxygenator device may be sensed. Acts 210, 212 and 214 may be repeated (at any suitable time interval) until it is determined in act 212 that the temperature sensed in act 212 is less than the threshold value. In some embodiments, the flow rate of the blower may not be decreased during each iteration of the feedback loop involving acts 210, 212 and 214. Rather, the flow rate may initially be decreased to a certain level upon detecting that the blower temperature exceeds a threshold value, and the sensed temperature can be monitored in subsequent iterations until the temperature is less than the threshold value without further decreasing the flow rate. In some embodiments, after it is determined in act 212 that the temperature exceeds the threshold value and the flow rate of the blower is decreased in act 214, subsequent iterations may compare the currently sensed temperature to a second threshold value that is less than the threshold value used in the initial determination. For instance, a first threshold value may be considered a maximum threshold value and may be used to initially detect that the temperature of the blower is too hot and should be decreased. Subsequently, a second threshold value lower than the first threshold value may be used to determine when the blower has sufficiently cooled down to be able to make further adjustments to the flow rate. In some embodiments, a range of temperature values including a minimum threshold value and a maximum threshold value may be specified, with the maximum threshold value being used for the initial determination in act 212 to reduce the flow rate and the minimum threshold value used in act 212 to determine that the blower has sufficiently cooled. It should be appreciated that in some embodiments, a single threshold value or more than two threshold values may be used.

As shown in FIG. 2, if it is determined in act 212 that the temperature sensed in act 210 is not greater than a threshold value, process 200 may proceed to act 216, where one or more actions may optionally be performed. For instance, if it is determined in act 212 that the sensed temperature exceeds a threshold value (e.g., a first threshold value) after which the flow rate of the blower is decreased in act 214, then after some time it is determined in act 212 that the sensed temperature does not exceed a threshold value (e.g., a second threshold value lower than the first threshold value), an indication (e.g., a visual indication, an audio indication) may be output on a user interface associated with the oxygenator device in act 216 to alert a user that the blower is sufficiently cooled. Providing such an indication to the user may inform the user that modifications to the flow rate (e.g., increasing the flow rate) may be performed. The user may then interact with the user interface to adjust the flow rate, if desired. In some embodiments, performing an action in act 216 may include automatically increasing the flow rate (e.g., to a flow rate previously set by a user) of the blower without requiring the user to manually adjust the flow rate. In this way, some embodiments of the present disclosure may enable automated temperature management of the blower of an oxygenator device by decreasing and/or increasing the flow rate of the blower as needed to prevent overheating while maintaining a flow rate set by a user of the device to the extent possible.

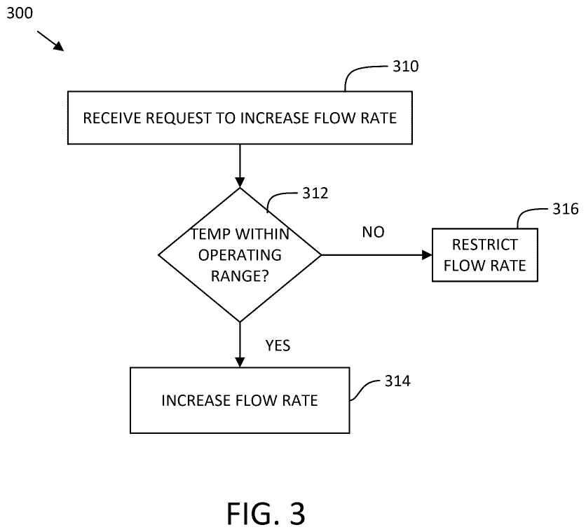

In some embodiments, a user's ability to modify the flow rate of a blower for an oxygenator device may be restricted based, at least in part, on a sensed temperature of the blower. FIG. 3 illustrates a flowchart of a process 300 for adjusting a flow rate of a blower for an oxygenator device in response to a user request, in accordance with some embodiments of the present disclosure. Process 300 may begin in act 310, where a request to increase the flow rate of the blower is received. For instance, a physician or other user may interact with a user interface of the oxygenator device to issue a request to increase the flow rate. Process 300 may then proceed to act 312, where it may be determined whether the temperature of the blower is within a particular operating range. In some embodiments, the determination in act 312 of whether the temperature of the blower is within the particular operating range may be based on a current temperature of the blower (e.g., as sensed by one or more temperature sensors coupled to a heat sink associated with the blower). In other embodiments, the determination in act 312 of whether the temperature of the blower is within the particular operating range may be based on a predicted temperature of the blower if the flow rate was increased in accordance with the request received in act 310. For instance, a controller of the oxygenator device (e.g., controller 122 shown in FIG. 1) may be configured to predict a temperature of the blower based, at least in part, on a current temperature of the blower and a flow rate included in the request to increase the flow rate. If it is determined based on the predicted temperature that the temperature of the blower will exceed a maximum threshold value in the operation range within some amount of time (e.g., 1 minute, 5 minutes, 10 minutes, 30 minutes, etc.) it may be determined in act 312 that the temperature does not fall within the operating range.

If it is determined in act 312 that the temperature (e.g., the current temperature and/or the predicted temperature) of the blower is within the operating range, process 300 may proceed to act 314, where the flow rate of the blower may be increased in response to the request received in act 310. If it is determined in act 312 that the temperature (e.g., the current temperature and/or the predicted temperature) of the blower is not within the operating range, process 300 may proceed to act 316, where the flow rate of the blower may be restricted such that the flow rate is not increased responsive to the request received in act 310. In some embodiments, an indication (e.g., a visual indication, an audio indication) reflecting whether the flow rate was increased in act 314 may be provided (e.g., on a user interface) to a user of the oxygenator device. In some embodiments, when the flow rate is restricted in act 316, an indication that the flow rate was restricted due to the temperature of the blower not being within the operating range, may be provided to the user. In this way, the blower temperature may stay within a temperature range that enables for safe operation of the oxygenator device, while informing the user about the reason for the flow rate restriction.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

1. An oxygenator device, comprising:
a blood oxygenator configured to add oxygen to and remove carbon dioxide from blood when present therein;
a blower configured to provide the oxygen to the blood oxygenator at a flow rate;
a temperature sensor configured to sense at a first time, a first temperature associated with the blower; and
at least one controller configured to adjust the flow rate of oxygen provided to the blood oxygenator by the blower based, at least in part, on the first temperature.

2. The oxygenator device of claim 1, further comprising:
a heat sink coupled to the blower, wherein the temperature sensor is coupled to the heat sink.

3. The oxygenator device of claim 1, wherein the temperature sensor is a thermistor.

4. The oxygenator device of claim 1, wherein the at least one controller is further configured to:
determine whether the first temperature is greater than a first threshold value; and
adjust the flow rate of oxygen provided by the blower by decreasing the flow rate when it is determined that the first temperature is greater than the first threshold value.

5. The oxygenator device of claim 4, wherein the temperature sensor is configured to sense at a second time, a second temperature associated with the blower, the second time being after the first time, and wherein the at least one controller is further configured to:
determine whether the second temperature is less than a second threshold value; and
control at least one operation of the oxygenator device when it is determined that the second temperature is less than the second threshold value.

6. The oxygenator device of claim 5, wherein the first threshold value and the second threshold value are a same value.

7. The oxygenator device of claim 5, wherein the second threshold value is less than the first threshold value.

8. The oxygenator device of claim 5, wherein the at least one controller is further configured to control at least one operation of the oxygenator device by outputting an indication on a user interface of the oxygenator device.

9. The oxygenator device of claim 8, wherein the indication includes an indication of the second temperature.

10. The oxygenator device of claim 8, wherein the indication includes an indication that the flow rate can be increased.

11. The oxygenator device of claim 5, wherein the at least one controller is further configured to control at least one operation of the oxygenator device by automatically increasing the flow rate.

12. The oxygenator device of claim 11, wherein automatically increasing the flow rate comprises automatically increasing the flow rate to the flow rate set at the first time.

13. The oxygenator device of claim 4, wherein decreasing the flow rate comprises lowering a maximum operating voltage provided to the blower.

14. The oxygenator device of claim 1, wherein the at least one controller is further configured to:
receive, via a user interface of the oxygenator device, a request to increase the flow rate to a new flow rate;
determine whether increasing the flow rate to the new flow rate is likely to result in a temperature of the blower exceeding a first threshold value within a predetermined amount of time; and
increase the flow rate to the new flow rate when it is determined that increasing the flow rate to the new flow rate is unlikely to result in the temperature of the blower exceeding the first threshold value within the predetermined amount of time.

15. The oxygenator device of claim 14, wherein the at least one controller is further configured to restrict increasing the flow rate to the new flow rate when it is determined that increasing the flow rate to the new flow rate is likely to result in the temperature of the blower exceeding the first threshold value within the predetermined amount of time.

16. An extracorporeal membrane oxygenator (ECMO) system including the oxygenator device of claim 1.

17. A controller for an oxygenator device, the controller configured to:
receive a first temperature associated with a blower of the oxygenator device, wherein the first temperature is sensed by a temperature sensor of the oxygenator device; and
adjust a flow rate of oxygen provided to a blood oxygenator by the blower based, at least in part, on the first temperature.

18-31. (canceled)

32. A method of controlling a flow rate of an oxygenator device, the method comprising:
receiving a first temperature associated with a blower of the oxygenator device, wherein the first temperature is sensed by a temperature sensor of the oxygenator device; and
adjusting a flow rate of oxygen provided to a blood oxygenator by the blower based, at least in part, on the first temperature.

33. The method of claim 32, further comprising:
determining whether the first temperature is greater than a first threshold value; and
adjusting the flow rate of oxygen provided by the blower by decreasing the flow rate when it is determined that the first temperature is greater than the first threshold value.

34. The method of claim 33, wherein the first temperature is received at a first time, the method further comprising:
receiving at a second time, a second temperature associated with the blower, the second time being after the first time;
determining whether the second temperature is less than a second threshold value; and
controlling at least one operation of the oxygenator device when it is determined that the second temperature is less than the second threshold value.

35-44. (canceled)
